SEQUENCE LISTING (2 sequences)

SEQ ID NO: 1 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 47 aa, Homo sapiens
ELRMSSSYPTGLADVKAGPAQTLIRPQDMKGASRSPEDSSPDAARIR

(19) United States
(12) Patent Application Publication
(10) Pub. No.: US 2010/0035275 A1
(43) Pub. Date: Feb. 11, 2010

(54) DIAGNOSIS AND RISK ASSESSMENT OF PANCREATIC DIABETES USING MR-PROADM

(75) Inventors: Andreas Bergmann, Berlin (DE); Nils Morgenthaler, Berlin (DE); Jana Papassotiriou, Berlin (DE); Joachim Struck, Berlin (DE)

Correspondence Address: CONNOLLY BOVE LODGE & HUTZ, LLP, P O BOX 2207, WILMINGTON, DE, 19899, US

(73) Assignee: Brahms Aktiengesellschaft, Henningsdorf (DE)

(21) Appl. No.: 12/514,194

(22) PCT Filed: Nov. 8, 2007
(86) PCT No.: PCT/DE2007/002018

§ 371 Date: Sep. 18, 2009

(30) Foreign Application Priority Data
Nov. 8, 2006 (DE) 10 2006 052 916.2

(51) Int. Cl. G01N 33/53; C12M 1/34

(52) U.S. Cl. 435/7.1; 435/287.1

(57) ABSTRACT

The invention relates to a method for diagnosis and/or risk assessment of pancreatic diabetes, in particular of diabetic sequelae, wherein a determination of the marker mid-regional proAdrenomedullin (MR-proADM: SEQ ID No. 2) or a partial peptide or fragment thereof or if contained in a marker combination (Panel, Cluster) is carried out on a patient under investigation. The invention further relates to a diagnostic device and a kit for carrying out said method.

The Invention relates to a method for the diagnosis and/or risk stratification of diabetes mellitus, particularly of diabetic sequelae, wherein a determination of the marker midregional proadrenomedullin (MR-proADM: SEQ ID No. 2) or a partial peptide or fragment thereof, or contained in a marker combination (panel, cluster), is carried out on a patient to be investigated. Furthermore, the invention relates to a diagnostic device and a kit for carrying out the method.

The diagnosis of diabetes mellitus is known for adrenomedullin (Garcia-Unzueta M T, Montalban C, Pesquera C, Berrazueta J R, Amado J A. Plasma adrenomedullin levels in type 1 diabetes. Relationship with clinical parameters. Diabetes Care. 21:999-1003, 1998, and Turk H M, Buyukberber S, Sevinc A, Ak G, Ates M, Sari R, Savil H, Cigli A. Relationship between plasma adrenomedullin levels and metabolic control, risk factors, and diabetic microanglopathy in patients with type 2 diabetes. Diabetes Care 23:864-7, 2000). However, it is disadvantageous that because of the lack of stability of the adrenomedullin, as well as its short lifetime in the plasma (Lewis L K, Smith M W, Yandle T G, Richards A M, Nicholls M G. Adrenomedullin (1-52) measured in human plasma by radioimmunoassay: plasma concentration, adsorption, and storage. Clin Chem. 44:571-7, 1998), no reliable diagnosis can take place. However, there is a need for presenting a reliable diagnosis of diabetes mellitus, or for undertaking a (risk) stratification, particularly with regard to further clinical decisions and, in particular, with regard to the degree of severity of diabetes mellitus or diabetic sequelae.

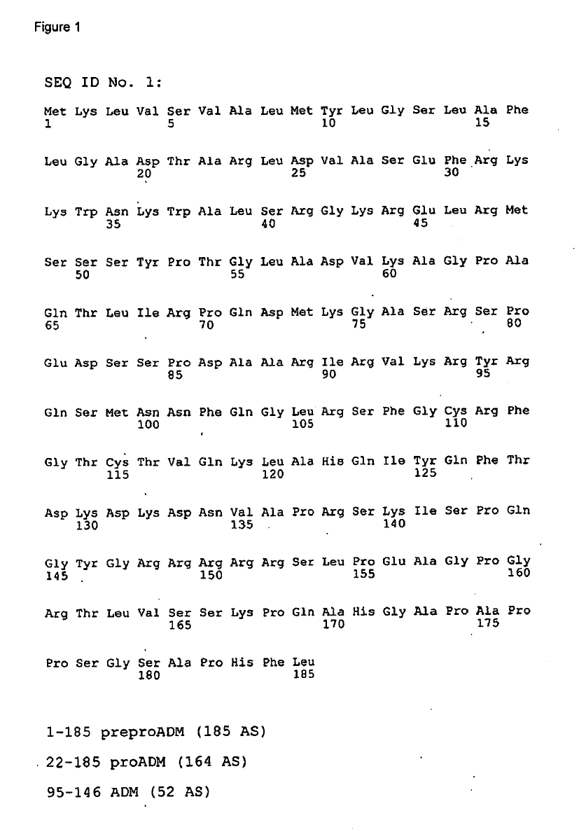

Furthermore, the proadrenomedullin (proADM) determination in diagnosis is described in the state of the art (EP0622458B1), particularly with regard to an Investigation of sepsis (EP1121600B1). Furthermore, another fragment of proadrenomedullin—namely what is called the midregional proadrenomedullin (MR-proADM: SEQ ID No. 2, also amino acid 45-92 of preproADM in SEQ ID No. 1 (FIG. 1)), is disclosed for diagnostic purposes in EP1488209B1. However, suitability of midregional proadrenomedullin (MR-proADM: SEQ ID No. 2) for the diagnosis of diabetes mellitus is not disclosed.

It is the task of the present invention to make available an improved method for the diagnosis and/or risk stratification of diabetes mellitus, particularly of diabetic sequelae.

This task is accomplished by means of a method for in vitro diagnosis and/or risk stratification of diabetes mellitus, wherein a determination of the marker midregional proadrenomedullin (MR-proADM: SEQ ID No. 2) or a partial peptide or fragment thereof, or contained in a marker combination (panel, cluster), is carried out in a patient to be investigated (referred to hereinafter as method according to the invention).

The term “risk stratification,” according to the invention, comprises finding diabetes patients, particularly those having diabetic sequelae, with the worse prognosis, for the purpose of intensive diagnosis and therapy/treatment (of sequelae) of diabetes mellitus, with the goal of allowing as advantageous a course of the diabetes mellitus as possible.

For this reason, it is particularly advantageous that a reliable diagnosis and/or risk stratification can take place by means of the method according to the invention. The method according to the invention allows clinical decisions that lead to a more rapid diagnosis, particularly of the diabetic sequelae. Such clinical decisions also comprise further treatment using medications, for the treatment or therapy of diabetes mellitus.

In another preferred embodiment of the method according to the invention, diagnosis and/or risk stratification take place for prognosis, for prophylaxis, for early detection and detection by means of differential diagnosis, for assessment of the degree of severity, and for assessing the course of diabetes mellitus as an accompaniment to therapy.

In another preferred embodiment of the method according to the invention, samples of bodily fluids, particularly blood, optionally whole blood, serum, or available plasma, are taken from the patient to be investigated, and the diagnosis takes place in vitro/ex vivo, i.e. outside of the human or animal body. The diagnosis and/or risk stratification can take place on the basis of the determination of the marker midregional proadrenomedullin (MR-proADM: SEQ ID No. 2) or partial peptides or fragments thereof, and Its amount that is present, or a change in amount, as compared with a reference, in at least one patient sample.

Within the scope of this invention, the term “diabetes mellitus,” particularly Type II diabetes mellitus (insulin-resistant), is understood to mean a chronic metabolic disease, where the production of insulin in the beta cells of the islets of Langerhans in the pancreas is disturbed, or insulin is present but cannot correctly act at its target location, the cell membranes. The results of this disturbed insulin production and effect are elevated blood sugar values (hyperglycemia). In the diabetic disease profile, a differentiation is made between prediabetes, in which a “disturbed glucose tolerance,” which can be detected by laboratory chemistry, occurs only in the end stage, and actual manifest diabetes mellitus. Insulin resistance stands at the beginning of the prediabetic illness phase. Almost at the same time, endothelial dysfunction already develops, along with hyperlipoproteinemia and hypertensive dysfunction of the cardiovascular system. The results of this risk constellation are furthermore artherosclerotic changes in the blood vessel walls (microangiopathy and macroanglopathy), as well as vascular complications as the result of microcirculation problems. Other sequelae and concomitant illnesses are diabetic retinopathy going as far as blindness, as well as nephropathy going as far as renal insufficiency, neuropathy, the diabetic foot syndrome, and cardiovascular complications. For this reason, the invention relates to the diagnosis and/or risk stratification of Type II diabetes mellitus, and its sequelae and concomitant illnesses, particularly endothelial dysfunction, hyperlipoproteinemia, hypertensive dysregulation of the cardiovascular system, diabetic retinopathy, nephropathy, renal insufficiency, neuropathy, diabetic foot syndrome, and cardiovascular complications.

All the aforementioned indications are furthermore described in Pschyrembel, De Gruyter, Berlin 2004, for example.

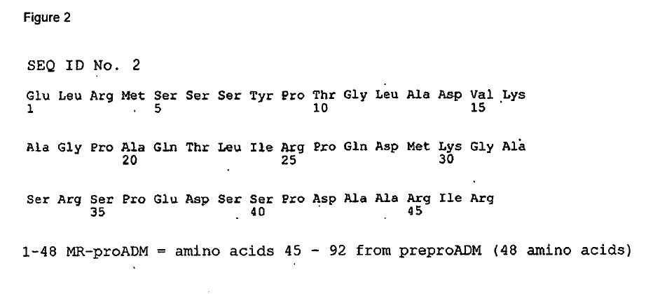

Within the scope of this invention, “midregional proadrenomedullin (MR-proADM: SEQ ID No. 2)” is understood to be a human protein or polypeptide having an amino acid sequence of 45-92 (position 45 is Glu, position 92 is Val) having the SEQ ID No. 1 (FIG. 1) of preproadrenomedullin (Kitamura K, Sakata J, Kangawa K, Kojima M, Matsuo H, Eto T. Cloning and characterization of cDNA encoding a precursor for human adrenomedullin. Biochem Biophys Res Commun 1993: 194:720-725), and/or amino acid sequence 148 having the SEQ ID No. 2 (FIG. 2). This fragment of proadrenomedullin is called “midregional proadrenomedullin (MR-proADM)” (EP 1488209B1), and demonstrates great plasma stability, which is particularly advantageous.

Furthermore, the ‘midregional proadrenomedullin’ according to the invention can demonstrate modifications such as glycolization, lip(o)idiation, or derivatization.

In another embodiment, the determination of midregional proadrenomedullin (MR-proADM: SEQ ID No. 2) can additionally take place with other markers, where the midregional proadrenomedullin (MR-proADM: SEQ ID No. 2) is contained in a marker combination (panel, cluster), specifically preferably those that already indicate diabetes mellitus. In another particular embodiment, this can also be a vascular marker that can indicate an endothelial dysfunction of the cardiovascular system that accompanies diabetes.

For this reason, the Invention relates to an embodiment of the method according to the invention where the determination is additionally carried out with at least one further marker selected from the group of inflammatory markers, vascular markers, in a patient to be investigated.

According to the invention, the inflammatory marker can be selected from at least one marker of the group of C-reactive protein (CRP), cytokines, such as TNF-alpha, for example, interleukins, such as IL-6, interleukin-1β, procalcitonin (1-116, 3-116), angiotensin II, endothelin-1, and adhesion molecules, such as VCAM or ICAM, and the vascular marker can be selected from at least one marker of the group of creatine kinase, myeloperoxidase, myoglobin, natriuretic protein, particularly ANP (or ANF), proANP, NT-proANP, BNP, proBNP, NT-proBNP, or a partial sequence thereof, in each instance, CRP. Furthermore, this term is also understood to mean (pro)hormones that regulate the cardiovascular system, particularly such as pro-gastrin-releasing peptide (proGRP), pro-endothelin-1, pro-leptin, pro-neuropeptide-Y, pro-somatostatin, pro-neuropeptide-YY, pro-opiomelanocortin, or a partial sequence thereof, in each instance.

In another embodiment, at least one diabetic marker/factor can additionally be determined. Diabetic markers/factors are, according to the invention, particularly those such as adiponectin, carbohydrates, fats, such as cholesterols (LDH) and others, Body Mass Index (BMI), age, blood pressure, HOMA-IR (Homeostasis Model Assessment-insulin Resistance index, for a determination see: Matthews D R, Hosker J P, Rudenski A S, Naylor B A, Treacher D F, Turner R C, Homeostasis Model Assessment: Insulin Resistance and B-cell Function from Fasting Plasma Glucose and Insulin Concentrations in Man. Diabetologia 28:412-419. 1985).

In another embodiment of the Invention, the method according to the invention can be carried out by means of parallel or simultaneous determinations of the markers (e.g. mufti-titer plates with 96 cavities and more); where the determinations are carried out on at least one patient sample.

Furthermore, the method according to the invention and its determinations can be carried out using an automated analysis device, particularly using a Kryptor (http://www.kryptor.net/).

In another embodiment, the method according to the Invention and Its determinations can be carried out by means of a rapid test (e.g. lateral flow test), whether using single-parameter or multi-parameter determinations.

Furthermore, the Invention relates to the use of midregional proadrenomedullin (MR-proADM: SEQ ID No. 2) or partial peptides or fragments thereof, or contained in a marker combination (panel, cluster), for in vitro diagnosis and/or risk stratification of diabetes mellitus, particularly Type II diabetes mellitus, and its sequelae and concomitant illnesses, as well as, in particular, taking the aforementioned embodiments into consideration. The marker combination can contain another suitable marker, if necessary.

Another task is making available a corresponding diagnostic device, or the use of such a device for carrying out the methods according to the Invention.

Within the scope of this invention, such a diagnostic device is particularly understood to be an array or assay (e.g. immune assay, ELISA, etc.), in the broadest sense a device for carrying out the method according to the invention.

The invention furthermore relates to a kit or the use of such a kit for in vitro diagnosis or risk stratification of diabetes mellitus, particularly Type II diabetes mellitus, and its sequelae and concomitant illnesses, where a determination of midregional proadrenomedullin (MR-proADM: SEQ ID No. 2) or partial peptides or fragments thereof, or contained in a marker combination (panel, cluster), is carried out in a patient to be investigated, particularly taking into consideration the aforementioned embodiments. Such detection reagents comprise antibodies, etc., for example.

The following examples and figures serve for a more detailed explanation of the invention, but without restricting the invention to these examples and figures.

### EXAMPLES

### Example 1

The MR-proADM assay was carried out according to Morgenthaler et al. (Morgenthaler N G, Struck J, Alonso C, Bergmann A. Measurement of midregional proadrenomedullin in plasma with an immunoluminometric assay. Clin Chem. 2005 October; 51(10):1823-9).

MR-proADM was determined in 100 healthy test subjects with undisturbed glucose tolerance, 60 patients who already had disturbed glucose tolerance, and 200 patients having manifest diabetes mellitus Type II (abbreviated as: “DM II”). These 200 patients were divided up into 100 diabetics not suffering from any sequelae, and 100 diabetics who were already suffering from sequelae, such as diabetic nephropathy and diabetic retinopathy.

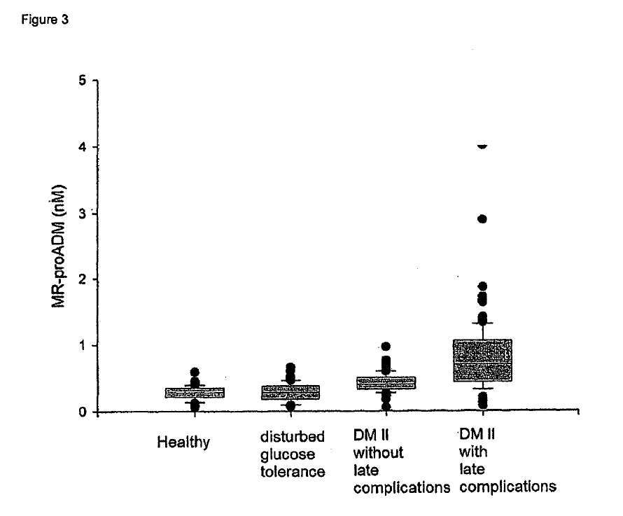

FIG. 3 shows a significant increase in the MR-proADM values with an increasing degree of severity of the DM II. The two groups of DM II patients, in particular, differ from the healthy controls and the patients having disturbed glucose tolerance. Surprisingly, the highest MR-proADM values were found in the DM II patients who already demonstrated diabetic sequelae.

Table 1 shows not only MR-proADM but also the parameters relevant to diabetes, such as glucose and HbAlc, in the groups, in each instance.

TABLE 1

| | | | Disturbed glucose | | DM II | | DM II | | |
|---|---|---|---|---|---|---|---|---|---|
| | Healthy | | tolerance | | without SK | | with SK | | p value |
| | (n = 100) | | (n = 60) | | (n = 100) | | (n = 100) | | (for trend) |
| Sex (% male) | 50 | | 32 | | 58 | | 55 | | 0.007 |
| Age (years) | 40 | (14) | 44 | (11) | 58 | (10) | 61 | (10) | <0.001 |
| Duration of illness (years) | — | | — | | 16 | (7) | 17 | (9) | 0.55 |
| Body Mass Index (kg/m²) | 22.9 | (3.7) | 24.1 | (4.2) | 25.8 | (4.2) | 26.1 | (4.5) | <0.001 |
| Systolic blood pressure | 121 | (14) | 126 | (22) | 136 | (17) | 147 | (21) | <0.001 |
| (mmHg) | | | | | | | | | |
| Diastolic blood pressure | 77 | (8) | 78 | (12) | 81 | (8) | 81 | (11) | 0.02 |
| (mmHg) | | | | | | | | | |
| Fasting plasma glucose | 4.9 | (0.3) | 5.9 | (0.4) | 7.0 | (1.2) | 8.5 | (3.5) | <0.001 |
| (mmol/L) | | | | | | | | | |
| HBA1c (%) | — | | — | | 7.8 | (1.3) | 8.1 | (1.6) | 0.07 |
| Midregional | 0.27 | (0.09) | 0.29 | (0.13) | 0.42 | (0.13) | 0.81 | (0.54) | <0.001 |
| proadrenomedullin | | | | | | | | | |
| (mmol/L) | | | | | | | | | |

(Late complications (SK) (syn.: sequelae))

The relationship between restricted microcirculation that is already present and MR-proADM values is also shown by a significant correlation (r=0.43, P=0.002) between the MR-proADM values in 50 patients having DM II and the circulation flow measured as resting forearm cutaneous micro-circulatory perfusion. (RCMP: A Enrique Caballero, Rola Saouaf, Subodh Arora, Su C Lim, Frank W LoGerfo, Edward S Horton, Aristidis Veves. Reactivity of the micro- and macro-circulation is impaired in those at risk for type 2 diabetes. Diabetes 48:1856-1862, 1999).

Figures:

FIG. 1 shows SEQ ID No. 1 of preproADM with the related partial sequences.

FIG. 2 shows SEQ ID No. 2 of MR-proADM.

FIG. 3 shows MR-proADM in healthy test subjects, patients having disturbed glucose tolerance, patients having Type II diabetes mellitus (DM II) without late complications (SK) (syn.: sequelae), and patients having DM II with late complications (syn.: sequelae).

1. Method for in vitro diagnosis and/or risk stratification of diabetes mellitus, comprising determining midregional proadrenomedullin (MR-proADM: SEQ ID No. 2) or partial peptides or fragments thereof in a patient to be investigated.

2. Method according to claim 1,
characterized in that in vitro diagnosis and/or risk stratification of Type II diabetes mellitus and its sequelae and concomitant illnesses, particularly endothelial dysfunction, hyperlipoproteinemia, hypertensive dysregulation of the cardiovascular system, diabetic retinopathy, nephropathy, renal insufficiency, neuropathy, diabetic foot syndrome, and cardiovascular complications, take place.

3. Method according to claim 1, further comprising determining at least one further marker selected from the group of inflammatory markers, vascular markers, and/or diabetic markers/factors, in a patient to be investigated.

4. Method according to claim 3, characterized in that the inflammatory marker is selected from at least one marker of the group of C-reactive protein (CRP), cytokines, such as TNF-alpha, for example, interleukins, such as IL-6, interleukin-1β, procalcitonin (1-116, 3-116), angiotensin II, endothelin-1.

5. Method according to claim 3, characterized in that the vascular marker is selected from at least one marker of the group of creatine kinase, myeloperoxidase, myoglobin, natriuretic protein, particularly ANP (or ANF), proANP, NT-proANP, BNP, proBNP, NT-proBNP, or (pro)hormones that regulate the cardiovascular system, such as pro-gastrin-releasing peptide (proGRP), pro-endothelin-1, pro-leptin, pro-neuropeptide-Y, pro-somatostatin, pro-neuropeptide-YY, pro-opiomelanocortin, or a partial sequence thereof, in each instance.

6. Method according to claim 3, characterized in that
parallel or simultaneous determinations of the markers are carried out.

7. Method according to claim 1, characterized in that
the determinations are carried out on at least one patient sample.

8. Method according to claim 1, characterized in that
the determinations are carried out using an automated analysis device.

9. Method according to claim 1, characterized in that the determinations are carried out by means of a rapid test.

10. Method according to claim 1, characterized in that the diagnosis is for the stratification of patients for clinical decisions related to treatment or therapy of diabetes mellitus.

11. Method according to claim 1, characterized in that the diagnosis and/or risk stratification takes place for prognosis, for prophylaxis, for early detection and detection by means of differential diagnosis, for assessment of the degree of severity, and for assessing the course of diabetes mellitus, particularly Type II diabetes mellitus, and its concomitant illnesses and sequelae, as an accompaniment to therapy.

12. (canceled)

13. Diagnostic device for carrying out a method according to claim 1.

14. Kit for in vitro diagnosis and/or risk stratification of diabetes mellitus, particularly Type II diabetes mellitus, and its concomitant illnesses and sequelae,
containing detection reagents for determining the marker midregional proadrenomedullin (MR-proADM: SEQ ID No. 2) or partial peptides or fragments thereof, or contained in a marker combination, wherein the marker combination contains other markers according to claim 3, and ancillary substances.
